(19) United States
(12) Patent Application Publication
(10) Pub. No.: US 2020/0323478 A1
(43) Pub. Date: Oct. 15, 2020

(54) INFORMATION PROCESSING DEVICE, STATE ACQUISITION PROGRAM, SERVER, AND INFORMATION PROCESSING METHOD

(71) Applicant: SHARP KABUSHIKI KAISHA, Sakai City, Osaka (JP)

(72) Inventors: MASAKI HAMAMOTO, Sakai City, Osaka (JP); AZUSA NAKANO, Sakai City, Osaka (JP); TETSUYA HAYASHI, Sakai City, Osaka (JP)

(21) Appl. No.: 16/754,024

(22) PCT Filed: Nov. 22, 2018
(86) PCT No.: PCT/JP2018/043153

§ 371 Date: Apr. 6, 2020

(30) Foreign Application Priority Data
Nov. 29, 2017 (JP) 2017-229027

(51) Int. Cl. A61B 5/00; A61B 5/024; A61B 5/16

(52) U.S. Cl. CPC A61B 5/4035; A61B 5/165; A61B 5/02405

(57) ABSTRACT

An information processing device is provided that includes a display and a processor configured to acquire vital data regarding a creature and cause the display to display an image in which data regarding the creature at a plurality of time points are plotted on a graph having an abscissa axis and an ordinate axis one of which represents an autonomic nervous balance of the creature and another represents a numerical value based on the vital data of a type that differs from the autonomic nervous balance of the creature.

### TECHNICAL FIELD

This application claims the benefit of Japanese Patent Application No. 2017-229027 filed Nov. 29, 2017, which is hereby incorporated by reference herein in its entirety.

The disclosure below relates to a technique for acquiring the psychological state or the physical state of a creature.

### BACKGROUND ART

There have been known techniques for acquiring the psychological state or the physical state of a creature. For example, Japanese Unexamined Patent Application Publication No. 2010-155166 (PTL 1) discloses a pulse wave diagnostic apparatus and a method for controlling a pulse wave diagnostic apparatus. According to PTL 1, the pulse wave diagnostic apparatus and the method for controlling a pulse wave diagnostic apparatus are characterized in that a pulse wave is detected by using a photoelectric sensor, and a fluctuation of the pulse wave is calculated from the detected pulse wave. More specifically, the method for controlling a pulse wave diagnostic apparatus includes a photoelectric pulse wave detecting unit and a pulse wave amplitude Poincare calculation unit. The photoelectric pulse wave detecting unit receives light transmitted through an artery or light scattered by an artery and detects a pulse wave. The pulse wave amplitude Poincare calculation unit calculates the pulse wave amplitude for each of beats of the pulse wave detected by the photoelectric pulse wave detecting unit and, thereafter, calculates, Poincare coordinates, the point of the pulse wave amplitude in the Cartesian coordinate plane formed by two consecutively calculated pulse wave amplitudes for each of the beats.

### CITATION LIST

### Patent Literature

PTL 1: Japanese Unexamined Patent Application Publication No. 2010-155166

### SUMMARY OF INVENTION

### Technical Problem

An object of the present disclosure is to provide an information processing device, a state acquisition program, a server, and an information processing method capable of detecting the psychological state or the physical state of a creature more accurately or more efficiently than before.

### Solution to Problem

According to an aspect of the present disclosure, an information processing device is provided that includes a display and a processor configured to acquire vital data regarding a creature and cause the display to display an image in which data regarding the creature at a plurality of time points are plotted on a graph having an abscissa axis and an ordinate axis one of which represents an autonomic nervous balance of the creature and another represents a numerical value based on the vital data of a type that differs from the autonomic nervous balance of the creature.

### Advantageous Effects of Invention

As described above, according to the present disclosure, an information processing device, a state acquisition program, a server, and an information processing method are provided capable of detecting the psychological state or the physical state of a creature more accurately or more efficiently than before.

### BRIEF DESCRIPTION OF DRAWINGS

FIG. 1 is a diagram illustrating the overall configuration of an information processing system 1 according to a first embodiment.

FIG. 2 is a diagram illustrating the functional configuration of the information processing system 1 according to the first embodiment.

FIG. 3 is a flowchart illustrating a processing procedure for calculating a first autonomic nervous balance in the information processing system 1 according to the first embodiment.

FIG. 4 illustrates an example of cardiac potential data and heartbeat intervals according to the first embodiment.

FIG. 5 is a diagram illustrating a correspondence relation table between a heartbeat interval R-R(n) and a next heartbeat interval R-R(n+1) according to the first embodiment.

FIG. 6 is an image diagram illustrating conversion. of a correspondence relation table 321A between a heartbeat interval R-R(n) and a next heartbeat interval R-R(n+1) into that based on an axis extending in a Y=X direction and an axis extending in a direction perpendicular to the Y=X direction, according to the first embodiment.

FIG. 7 illustrates a table denoting, as a first autonomic nervous balance, a rough standard values of the standard deviation on the Y=X axis and the standard deviation on the axis perpendicular to Y=X for each of the psychological states or physical states of a dog according to the first embodiment.

FIG. 8 illustrates a Poincare plot for a dog in an excited state, according to the first embodiment.

FIG. 9 illustrates a Poincare plot for a dog in a normal state in which breathing of the dog is stable, according to the first embodiment.

FIG. 10 illustrates a Poincare plot for a dog in a normal state, according to the first embodiment.

FIG. 11 illustrates a Poincare plot for a dog in a resting state according to the first embodiment.

FIG. 12 is a flowchart illustrating a processing procedure for calculating a second autonomic nervous balance in the information processing system 1 according to the first embodiment.

FIG. 13 illustrates a table denoting rough standard values of the standard deviation on the Y=X axis, the standard deviation on the axis perpendicular to Y=X, the product of the standard deviations representing a second autonomic nervous balance, and the ratio of the standard deviations for each of the psychological states or physical states of a dog according to the first embodiment.

FIG. 14 is a flowchart illustrating a first processing procedure for calculating a respiratory rate in the information processing system 1 according to the first embodiment.

FIG. 15 illustrates an example of a relationship between a heartbeat detection time and a heartbeat interval according to the first embodiment.

FIG. 16 illustrates an example of a power spectrum distribution according to the first embodiment.

FIG. 17 illustrates an example of RRI fluctuation and power spectrum distribution after spline interpolation when a dog is resting according to the first embodiment.

FIG. 18 illustrates an example of RRI fluctuation and power spectrum distribution after spline interpolation when a dog is excited according to the first embodiment.

FIG. 19 illustrates an example of the effect of a method for acquiring a respiratory rate according to the first embodiment.

FIG. 20 is a flowchart illustrating a second processing procedure for processing the respiratory rate in the information processing system 1 according to the first embodiment.

FIG. 21 is an image diagram illustrating an output graph according to the first embodiment.

FIG. 22 is a flowchart illustrating a processing procedure for drawing a diagnostic graph in the information processing system 1 according to the first embodiment.

FIG. 23 is an image diagram illustrating a first diagnostic graph according to a second embodiment.

FIG. 24 is an image diagram illustrating a second diagnostic graph according to the second embodiment.

FIG. 25 is an image diagram illustrating a third diagnostic graph according to the second embodiment.

FIG. 26 is an image diagram illustrating a diagnostic graph according to a fourth embodiment.

FIG. 27 is an image diagram illustrating a first diagnostic graph according to a fifth embodiment.

FIG. 28 is an image diagram illustrating a second diagnostic graph according to the fifth embodiment.

FIG. 29 is a diagram illustrating the functional configuration of a first information processing system 1 according to a sixth embodiment.

FIG. 30 is a diagram illustrating the functional configuration of a second information processing system 1 according to the sixth embodiment.

FIG. 31 is a diagram illustrating the functional configuration of a third information processing system 1 according to the sixth embodiment.

FIG. 32 is a diagram illustrating the functional configuration of a fourth information processing system 1 according to the sixth embodiment.

### DESCRIPTION OF EMBODIMENTS

Embodiments of the present disclosure are described below with reference to the accompanying drawings. In the following description, the same reference numerals are used throughout to designate the same parts. Their names and functions are the same. Therefore, detailed description of the part is not repeated.

### First Embodiment

### Overall Configuration of Information Processing System

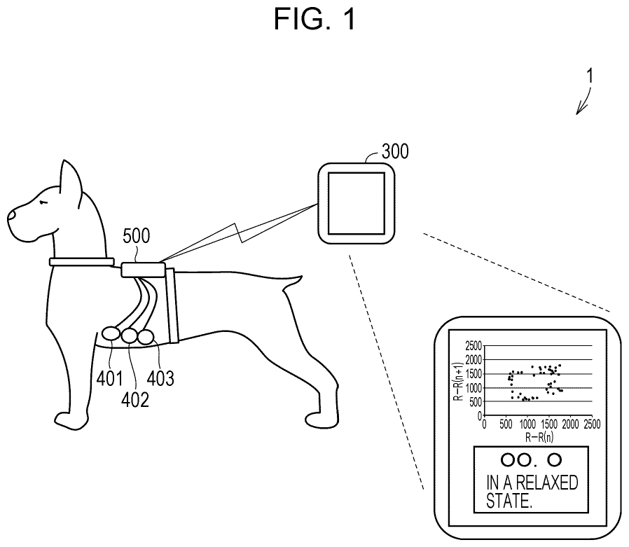

The overall configuration of an information processing system 1 according to the present embodiment is described first with reference to FIG. 1. FIG. 1 is a diagram illustrating the overall configuration of the information processing system 1 according to the present embodiment. Note that description below is given focusing on the case where the state of a dog, which is typical one of creatures, with respiratory arrhythmia is determined.

The information processing system 1 according to the present embodiment includes, but not limited to, electrodes 401, 402, and 403 attached to the chest of a dog to acquire the cardiac potential, a signal processing device 500 for processing cardiac potential signals, and a diagnostic terminal 300 capable of communicating with the signal processing device 500.

It is desirable that the electrodes 401, 402, and 403 for acquiring a cardiac potential be attached to the positions on the chest so as to sandwich the heart. For example, the electrodes 401, 402, and 403 can be attached to the paw pads of the two front legs (or the front leg and rear leg), where there is little fur. Alternatively, it is desirable that the fur be cut at the positions or that the electrodes have, for example, gel attached thereto or have a protruding structure so as to be in contact with the skin even when there is the fur. Still alternatively, it is desirable that when there is the fur, the cardiac potential be induced via a capacitive material in a non-contact manner. In this manner, the cardiac potential can be acquired even for a creature with a skin of fur, such as a dog. According to the present embodiment, the configuration including three electrodes 401, 402, and 403 is used. However, it is only required that the number of electrodes is two or greater. That is, the configuration using a greater number of electrodes can be used.

### Functional Configuration and Processing Procedure for Information Processing System

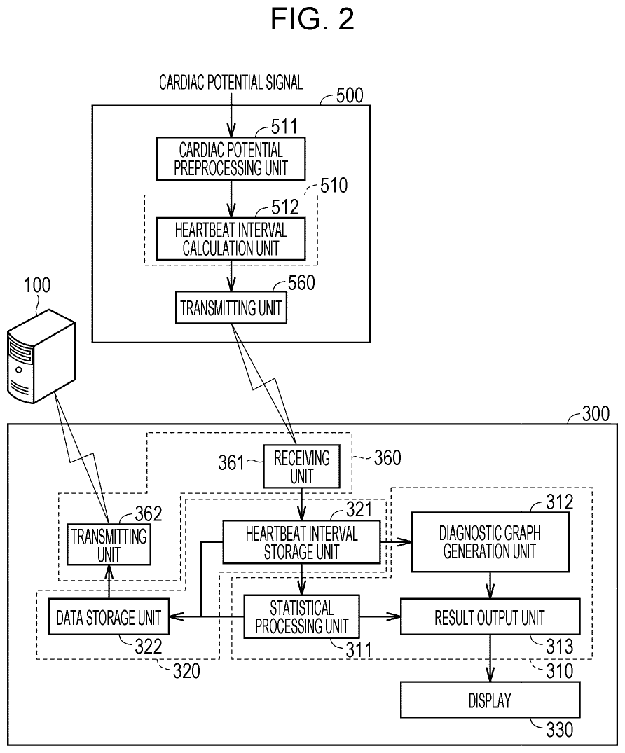

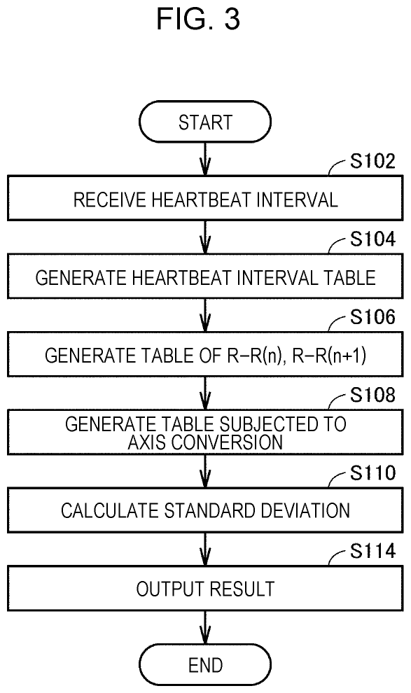

The functional configuration and the processing procedure for the information processing system 1 according to the present embodiment are described below with reference to FIG. 2 and FIG. 3. FIG. 2 is a diagram illustrating the functional configuration of the information processing system 1 according to the present embodiment. FIG. 3 is a flowchart illustrating the processing procedure using the information processing system 1 according to the present embodiment.

The configuration of the signal processing device 500 of the information processing system 1 is described first. The signal processing device 500 includes a cardiac potential preprocessing unit 511, a heartbeat interval calculation unit 512, and a transmitting unit 560.

The cardiac potential preprocessing unit 511 includes a filter and an amplifier. The cardiac potential preprocessing unit 511 converts the cardiac potential signals sent from the electrodes 401, 402, and 403 into heartbeat data and transfers the heartbeat data to the heartbeat interval calculation unit 512.

More specifically, the cardiac potential preprocessing unit 511 includes filter devices, such as a high-pass filter and a low-pass filter, an amplifier device including an operational amplifier, and an A/D conversion device that converts a cardiac potential analog signal into a digital signal. Note that the filter device and the amplifier device may be implemented in the form of software. Furthermore, it is desirable that the A/D conversion device perform sampling at a period and with accuracy such that a difference in fluctuation amount of a heartbeat interval is recognizable. That is, it is desirable that a cardiac potential signal be acquired at a frequency of 25 Hz or higher. For example, according to the present embodiment, sampling of a cardiac potential signal is performed at 100 Hz. By increasing the sampling frequency, the amount of fluctuation of the heartbeat interval can be accurately detected.

The heartbeat interval calculation unit 512 is implemented by, for example, a CPU (Central Processing Unit) 510 executing a program in a memory. The heartbeat interval calculation unit 512 sequentially calculates heartbeat intervals on the basis of the heartbeat data. More specifically, the heartbeat interval calculation unit 512 detects the peak signals (R waves) in the cardiac potentials by, for example, a threshold detection method and calculates an interval (time) between the peaks of the cardiac potentials. In addition to using the above method, the heartbeat interval may be calculated by a method for deriving a period using an autocorrelation function or a method using a rectangular wave correlation trigger.

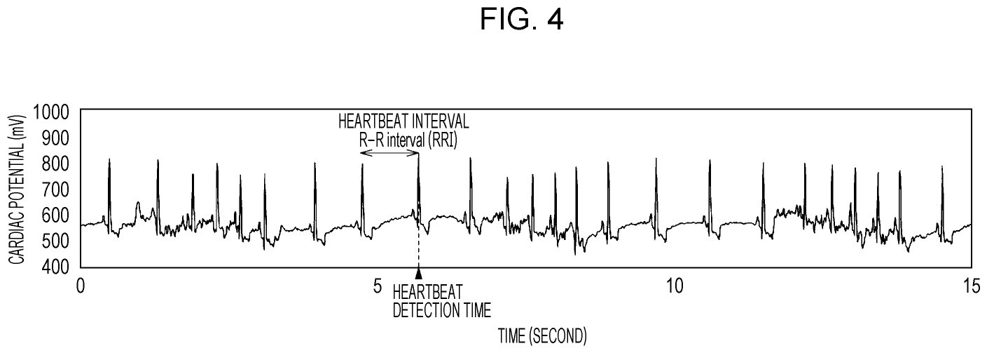

According to the present embodiment, as illustrated in FIG. 4, the heartbeat interval calculation unit 512 continuously calculates a heartbeat interval for the cardiac potential signal that is continuously input. The heartbeat interval calculation unit 512 transmits the calculated heartbeat interval and the raw heartbeat data to the diagnostic terminal 300 via the transmitting unit 560. Note that the transmitting unit 560 is implemented by a communication interface including, for example, an antenna and a connector.

The configuration of the diagnostic terminal 300 is described below. The diagnostic terminal 300 includes a receiving unit 361, a heartbeat interval storage unit 321, a statistical processing unit 311, a diagnostic graph generation unit 312, a result output unit 313, a display 330, a data storage unit 322, and a transmitting unit 362.

First, the receiving unit 361 and the transmitting unit 362 are implemented by a communication interface 360 including, for example, an antenna and a connector. The receiving unit 361 receives data indicating the heartbeat interval from the signal processing device 500 (step S102).

The heartbeat interval storage unit 321 includes a variety of memories 320 and the like and stores data received from the signal processing device 500. According to the present embodiment, a CPU 310 sequentially stores, in the memory 320, the heartbeat intervals received via the communication interface 360 in the form of a heartbeat interval table (step S104). However, these data may be stored in the memory 320 of the diagnostic terminal 300 or may be stored in another device accessible to the diagnostic terminal 300.

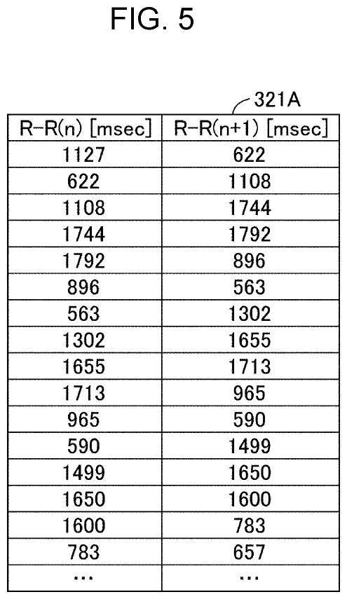

The statistical processing unit 311, the diagnostic graph generation unit 312, and the result output unit 313 are implemented by, for example, the CPU 310 executing a program in the memory 320. The statistical processing unit 311 reads the heartbeat interval data from the heartbeat interval storage unit 321 at predetermined time intervals that is required to determine the state (for example, at 1-minute intervals, at 10-minute intervals, or 1-hour intervals). Thus, as illustrated in FIG. 5, the statistical processing unit 311 generates a correspondence relation table 321A between a heartbeat interval R-R(n) and the next heartbeat interval R-R(n+1) (step S106). The heartbeat interval is calculated, for example, in the unit of msec (millisecond), as illustrated in the figure.

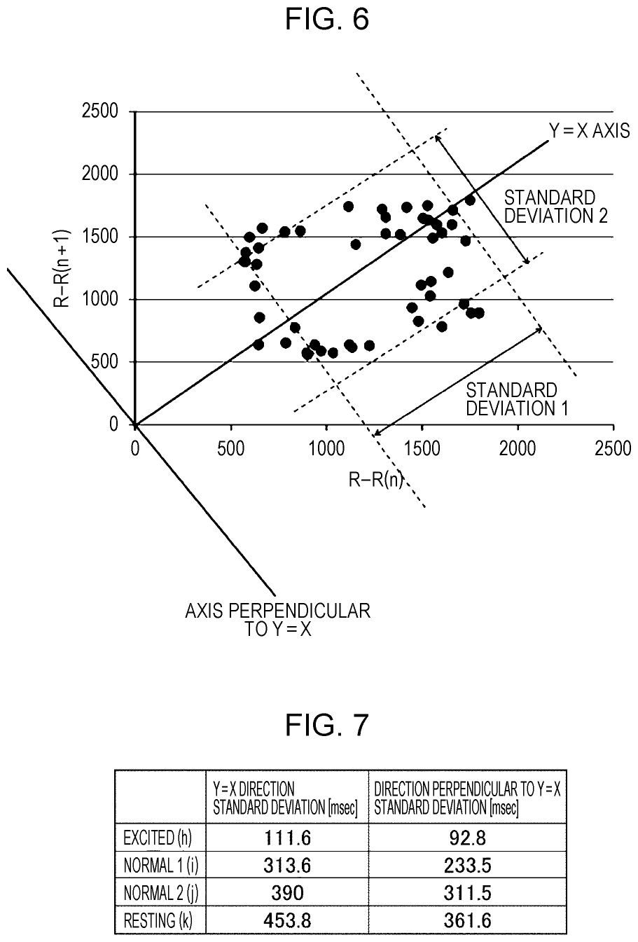

As illustrated in FIG. 6, the statistical processing unit 311 converts the numerical values in the correspondence relation table between the heartbeat interval R-R(n) and the next heartbeat interval R-R(n+1) into numerical values on the axis extending in the Y=X direction and numerical values on the axis extending in the direction perpendicular to the axis extending in the Y=X direction (step S108).

The statistical processing unit 311 calculates, as a numerical value representing the autonomic nervous balance, the standard deviation of a numerical sequence that constitutes each of the axes after the axis conversion (step S110). Note that the statistical processing unit 311 may calculate only the standard deviation. on the Y=X axis, may calculate only the standard deviation on the axis perpendicular to Y=X, or may calculate the standard deviations on both axes. FIG. 7 is a table denoting the rough standard values of the standard deviation on the Y=X axis and the standard deviation on the axis perpendicular to Y=X for each of the psychological or the physical states of the dog.

Note that the statistical processing unit 311 may identify the axis that maximizes the variance by using a method such as principal component analysis. Thereafter, the statistical processing unit 311 may calculate the standard deviations on the identified axis and an axis perpendicular to the identified axis. Alternatively, the statistical processing unit 311 may calculate the standard deviations on the X-axis and the Y-axis without performing the axis conversion. If the direction in which the variance increases is each of the X-axis direction and the Y-axis direction, the variation state of the heartbeat intervals in a Poincare plot can be evaluated without performing axis conversion by calculating the standard deviation on each of the X-axis and the Y-axis. In this case, since the need for performing axis conversion is eliminated, the amount of calculation can be reduced.

The result output unit 313 causes an output device, such as the display 330 or a loud loudspeaker, of the diagnostic terminal 300 or an external output device to display the standard deviation or output a speech message (step S114). More specifically, the result output unit 313 nay output only the standard deviation on the Y=X axis, may output only the standard deviation on the axis perpendicular to Y=X, or may output the standard deviations on both axes. Alternatively, the result output unit 313 may output only the smaller or larger of the standard deviations.

By calculating the standard deviation, it is possible to evaluate the variability of the heartbeat intervals in a Poincare plot of the heartbeat interval R-R(n) on one axis and the heartbeat interval R-R(n+1) on the other axis. Herein, the variability of the heartbeat intervals is regarded as the degree of autonomic nervous balance. Note that as described below, the numerical value representing the autonomic nervous balance is not limited to the standard deviation after axis conversion.

According to the present embodiment, the CPU 310 performs the calculation illustrated in FIG. 3 at predetermined time intervals, for example, at several minute intervals, and accumulates the result of calculation in a database in the memory 320 to generate a diagnostic graph (described below).

Although described in detail below, the information processing system 1 according to the present embodiment may include a server 100 that allows the diagnostic terminal 300 to communicate therewith, as illustrated in FIG. 2. In this case, the CPU 310 serving as the result output unit 313 accumulates the standard deviation and the relation table in the data storage unit 322 or transmits the standard deviation and the relation table to the server 100 via the Internet or the like by using the transmitting unit 362. In this manner, the current output result can be used to detect the short-term or long-term stress state of the target to be observed.

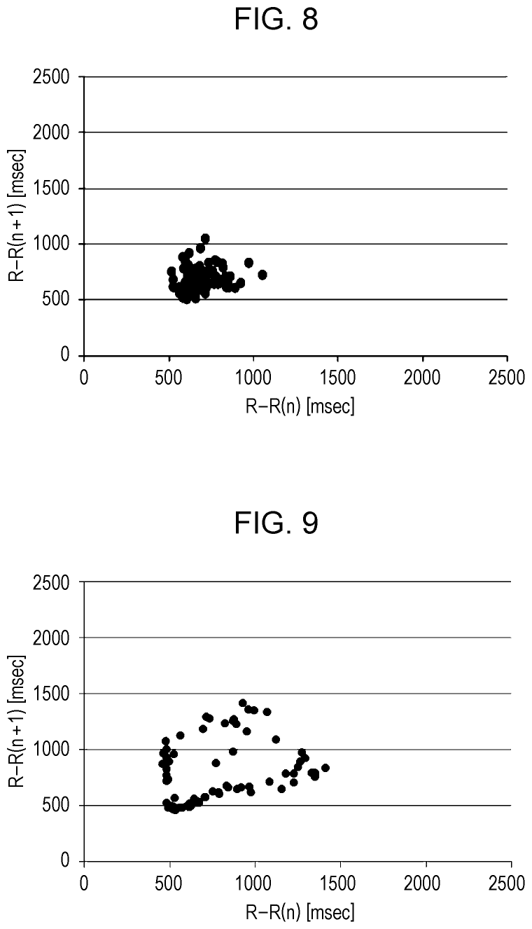

According to the present embodiment, separately from step S108, the diagnostic graph generation unit 312 obtains, from the correspondence relation table illustrated in FIG. 5, the data of the heartbeat interval R-R(n) and the next heartbeat interval R-R(n+1) in the range used for standard deviation calculation and generates a Poincare plot diagram illustrated in FIGS. 8 to 11.

Thereafter, the result output unit 313 causes an output device, such as a display of the diagnostic terminal 300 or an external display, to display the generated Poincare plot diagram. Note that the diagnostic graph generation unit 312 may generate and output a Poincare plot diagram subjected to axis conversion by using the result of step S108.

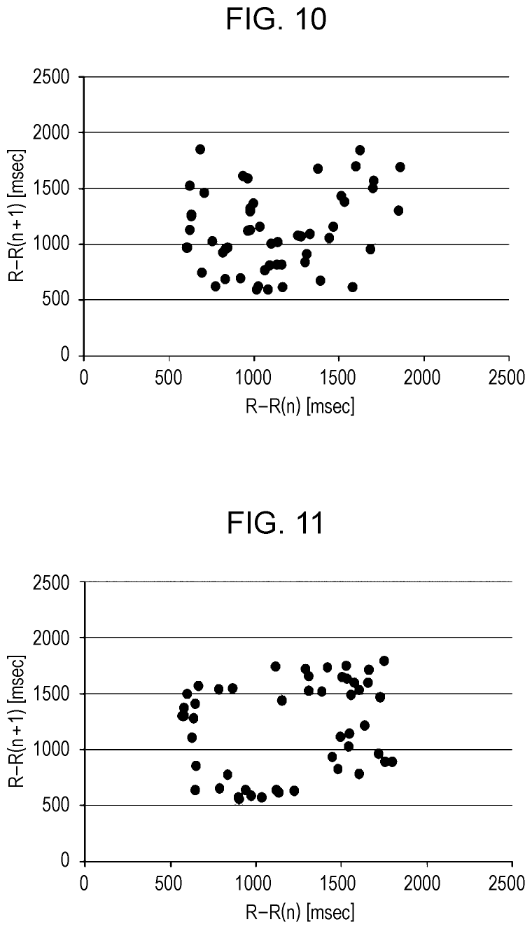

A Poincare plot diagram is described below. FIG. 8 is a Poincare plot diagram for a dog in as excited state according to the present embodiment. FIG. 9 is a Poincare plot diagram for the dog in a normal state in which breathing of the dog is stable according to the present embodiment. FIG. 10 is a Poincare plot diagram of the dog in a normal state according to the present embodiment. FIG. 11 is a Poincare plot diagram for the dog in a resting state according to the present embodiment.

First, in the case of a creature, such as a dog, with a respiratory arrhythmia, the heart rate of the dog in an excited state illustrated in FIG. 8 increases (the heartbeat interval decreases) and the fluctuation of the heartbeat interval decreases so that the points in the plot gather around a certain place.

In addition, in the normal state in which the breathing is stable as illustrated in FIG. 9, the heart rate is not so small as in the resting state (the spread of the points in the plot is not so large as in the resting state). However, there is an area with few plots (a blank space of a hole) around the center of the distribution of the points in the plot. The reason why such a shape is formed is that the heartbeat of the dog is greatly influenced by respiration and, thus, variation in heartbeats periodically changes (respiratory arrhythmia). For this reason, although the dog does not have a relaxed and gentle heartbeat, there is a blank space because the respiration is stably maintained.

In addition, in the normal state as illustrated in FIG. 10, the heartbeat fluctuates and, thus, the variation becomes large (the plotted dots spread), but the plotted dots are scattered.

In addition, in the resting state illustrated in FIG. 11, the interval between heartbeats increases because the dog is relaxed, and the dog is significantly influenced by respiratory arrhythmia. Accordingly, the plotted dots are largely spread into a shape close to a circular shape or rectangular shape or a shape close to a triangle. In any of these shapes, a blank portion is formed around the center of the distribution of the plotted dots in the Poincare plot when the dog is in the resting state.

As described above, according to the present embodiment, it is possible to indirectly estimate, on the basis of the result of calculation, the size and shape of the spread of distribution of the plotted dots in the Poincare plot and whether there are a small or large number of the plotted dots in the center portion of the distribution. As a result, the psychological state or the physical state of a creature can be estimated. Then, as described above, the statistical processing unit 311 calculates, as a numerical value representing the autonomic nervous balance, the degree of variation of the Poincare plot, that is, the standard deviation of the heartbeat intervals.

### Another Form of Numerical value of Autonomic Nervous Balance

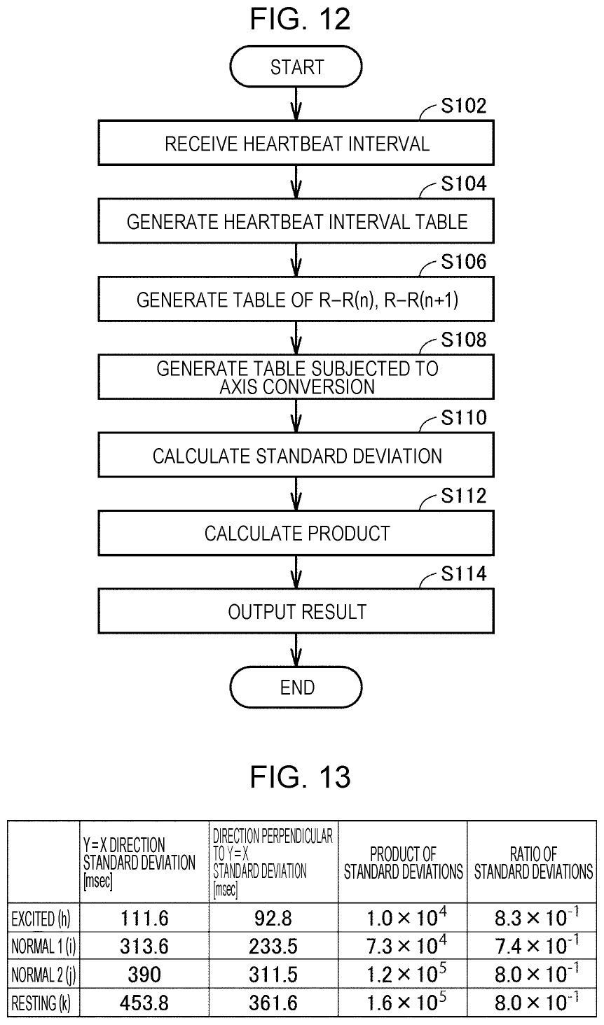

According to the above embodiment, the diagnostic terminal 300 outputs the standard deviation on the Y=X. axis or the standard deviation on the axis perpendicular to Y=X of the Poincare plot. However, the product of these two standard deviations may be calculated as a numerical value representing the autonomic nervous balance. The processing procedure using the information processing system 1 according to the present embodiment is described below with reference to FIG. 12.

FIG. 12 is a flowchart illustrating a processing procedure using the information processing system 1 according to the present embodiment. Steps S102 to S108 are the same as those in FIG. 3 and, thus, description thereof is not repeated here.

The CPU 310 serving as the statistical processing unit 311 calculates the standard deviation for each of the axes after the axis conversion (step S110). Note that the statistical processing unit 311 may identify the axis with the maximum variance and calculate the standard deviation on the identified axis and the standard deviation on the axis perpendicular to the identified axis.

Thereafter, the statistical processing unit 311 calculates, as a numerical value representing the autonomic nervous balance, the product of the two standard deviations or the square root of the product, for example (step S112).

The result output unit 313 causes an output device, such as a display or a loudspeaker, of the diagnostic terminal 300 or an external output device to display the product of standard deviations or the square root of the product or output a speech message (step S114). More specifically, the result output unit 313 may output the standard deviation on the Y=X axis, the standard deviation on the Y=−X axis, the product of the two standard deviations or the square root of the product, and the like.

FIG. 13 illustrates a table denoting rough standard values of the standard deviation on the Y=X axis, the standard deviation on the axis perpendicular to Y=X, the product of the standard deviations or the square root of the product as a numerical value representing an autonomic nervous balance, for example, and the ratio of the standard deviations for each of the psychological states or physical states of a dog.

By calculating the product of the standard deviations, it is possible to evaluate the size and shape of the spread of the heartbeat interval distribution in the Poincare plot of the heartbeat interval R-R(n) on one axis and the heartbeat interval R-R(n+1) on the other axis and the state of variability of the heartbeat intervals (e.g., the heartbeat intervals are uniformly distributed, or there is a blank space around the center of the distribution). In addition, the state of variability can be effectively evaluated when a change occurs in only the size while maintaining the aspect ratio or when the state of variability in the center portion changes although the area of the spread in distribution remains unchanged.

In this case, as in the above-described case, the result output unit 313 stores, in the data storage unit 322, the standard deviation, one of the product of the standard deviations and the square root of the product, the correspondence relation table, and the like or transmit these data to the server 100 via, for example, the Internet by using the transmitting unit 362. In this manner, the current output result can be used to detect the short-term or long-term stress state of a target to be observed.

The statistical processing unit 311 calculates the product of the standard deviations on the two axes or the square root of the product. However, the statistical processing unit 311 may calculate the product of the standard deviations on three or more axes or the power root of the product.

The CPU 310 performs the calculation illustrated in FIG. 12 at predetermined time intervals, for example, at several minute intervals, and accumulates the result of calculation in a database in the memory 320 to generate a diagnostic graph (described below).

### Technique for Calculating Respiratory Rate

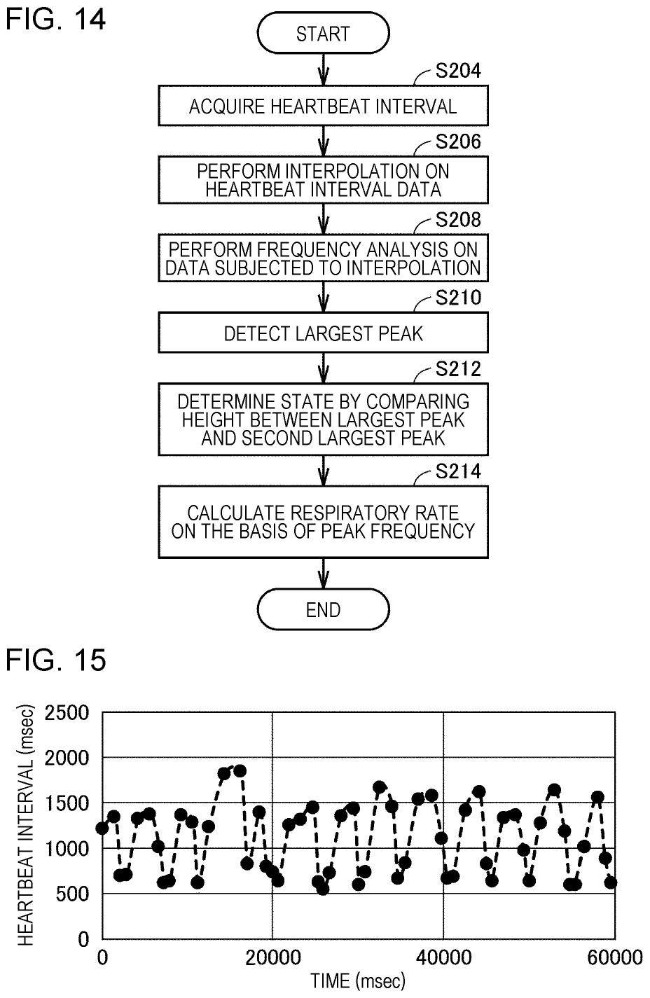

The CPU 310 of the diagnostic terminal 300 according to the present embodiment may calculate the respiratory rate of the target creature in addition to the information indicating the autonomic nervous balance of the target creature. Referring to FIG. 14, the CPU 310 of the diagnostic terminal 300 performs, for example, the processing described below by executing a program stored in the memory 320.

The CPU 310 acquires the heartbeat intervals Illustrated in FIG. 4 (step S204). As illustrated in FIG. 15, the CPU 310 mathematically performs interpolation (e.g., line interpolation) or the relationship between the heartbeat detection time and the heartbeat interval for one minute (step S206). More specifically, The CPU 310 detects peak signals (R wave) of cardiac potential by using, for example, a threshold detection method and calculates an interval (time) between the peaks of The cardiac potential. In addition to using the heartbeat interval calculation. method described above, the heartbeat interval may be calculated by a method for deriving a period using an autocorrelation function or a method using a rectangular wave correlation trigger.

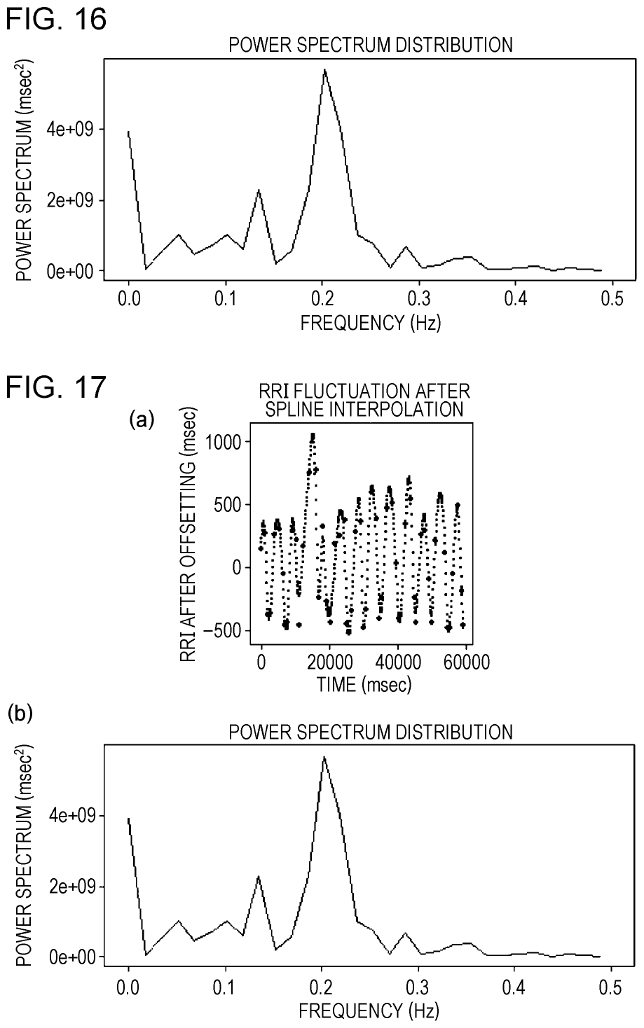

Thereafter, the CPU 310 performs frequency analysis on an obtained function illustrated in FIG. 16 (step S208).

The CPU 310 identifies the largest peak of the power spectrum in an arbitrarily determined frequency range (for example, a range between 0.05 Hz and 0.5 Hz) of the power spectrum distribution (illustrated in FIG. 16) obtained by the frequency analysis (step S210). Here, as an example, if the ratio of the largest peak to the second largest peak is larger than an arbitrarily determined threshold (for example, three times), the CPU 310 determines that the measurement is in a “measurable state”.

More specifically, for example, the RRI fluctuation after spline interpolation for a dog relaxing in a quiet indoor room is illustrated in FIG. 17(a). The power spectrum distribution in this case is illustrated in FIG. 17(b), and the ratio of the largest peak to the second largest peak is larger than an arbitrarily determined threshold (for example, three times). Thus, the CPU 310 determines that the state is a “measurable state”.

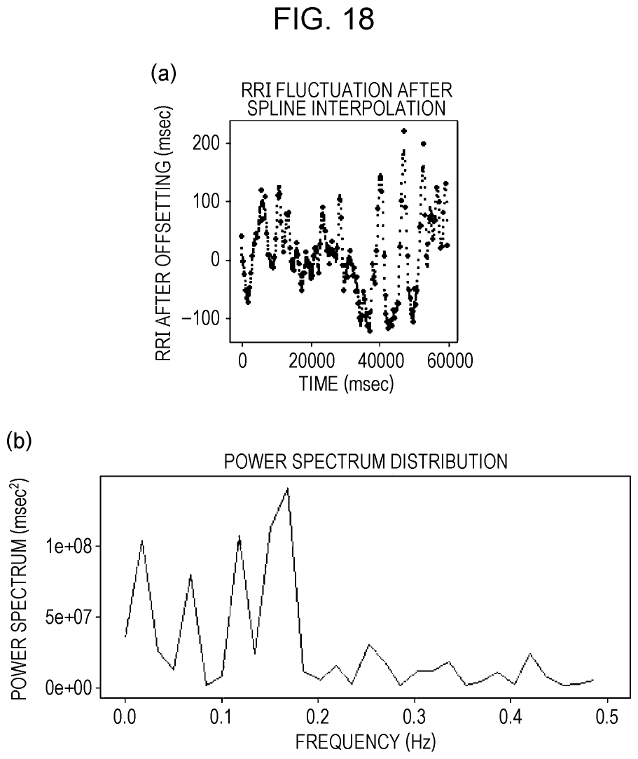

In contrast, for example, the RRI fluctuation after spline interpolation for the dog that is restless in a noisy outdoor environment is illustrated in FIG. 18(a). The power spectrum distribution in this case is illustrated in FIG. 18(b), and the ratio of the largest peak to the second largest peak is not larger than an arbitrarily determined threshold (for example, three times). Thus, the CPU 310 determines that the state is an “unmeasurable state”.

Upon determining that the state is an “unmeasurable state”, the CPU 310 repeats the processing from step S106 for another time point on the basis of the heartbeat interval already acquired by the signal processing device 500.

Upon determining that the state is a “measurable state”, the CPU 310 detects a variety of vital data. For example, the CPU 310 uses, as a respiratory frequency, the largest beak in an arbitrarily determined frequency range (for example, a range of 0.05 Hz to 0.5 Hz) in the frequency analysis and calculates the reciprocal. In this way, the CPU 310 calculates the respiratory rate.

The CPU 310 displays the number of breaths per unit time and outputs a speech message via, for example, the display 330, a loudspeaker 370, or the communication interface 360 for transmitting data to the outside. In addition, the CPU 310 performs the calculation illustrated in FIG. 14 at predetermined time intervals, for example, at several minute intervals, and accumulates the result of calculation in the database in the memory 320 to generate a diagnostic graph (described below).

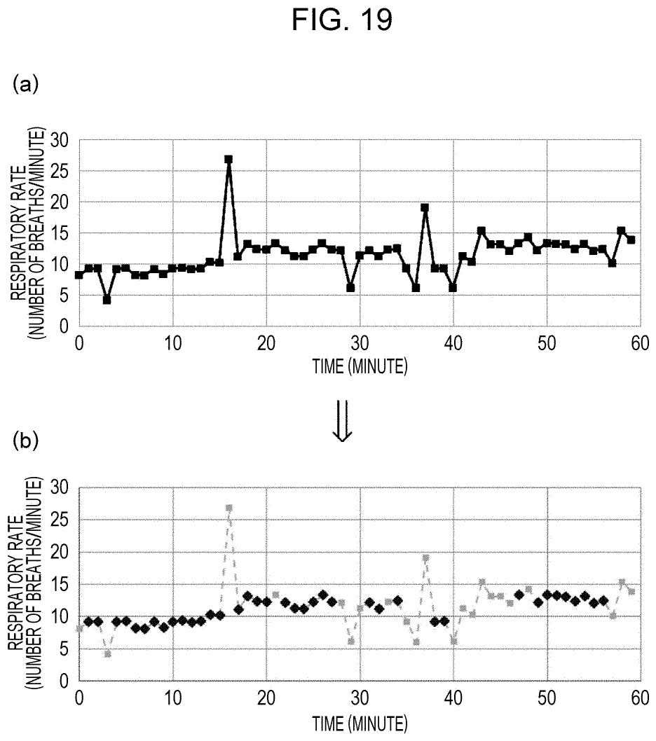

According to the present embodiment, the CPU 310 uses, as the frequency of breathing, the largest peak frequency in the frequency analysis and calculate the reciprocal of the frequency. In this way, the CPU 310 calculates the respiratory rate. FIG. 19 illustrates the result of the respiratory rate measurement for 60 minutes. It the state is not determined, the result of measurement can be output every minute as illustrated in FIG. 19(a). However, the result of measurement includes the results of measurements in various states. In addition, it is difficult to obtain sufficient accuracy. In contrast, by not calculating the data at the time when the state is determined as an “unmeasurable state”, it is possible to calculate the respiratory rates as illustrated in FIG. 19(b). Thus, only the respiratory rates in as appropriate state can be obtained.

More specifically, accumulating vital data has medical significance, but it is necessary to compare and analyze data measured in a certain environment (for example, during rest). In particular, to reliably record vital data when comparing data over a long period of time or when a subject (e.g., a dog) cannot maintain. a certain state by themselves, it is necessary to determine the state of the subject. In particular, since the respiratory rate fluctuates under voluntary control, it is difficult for the subject to consciously generate a measurable state. At present, there is no established means for automatically determining whether measurement is available.

However, the state of the subject can be determined by analyzing the measurement data (e.g., a cardiac potential signal), and vital data (e.g., the respiratory rate derived from the cardiac potential signal) can be calculated on the basis of the result of determination of the state. Thereafter, the vital data can be recorded. In particular, as means for determining the state, a determination is made as to “whether an appropriate state has been maintained for a certain period of time (e.g., one minute) during the measurement”. At this time, the criterion of determination as to “whether an appropriate state has been maintained” is defined from the variability cycle caused by respiration through, for example, heart rate variability analysis. In terms of an animal, such as a dog, even when an animal does not move, the heart rate and the respiratory rate change. For this reason, according to the above-described criterion of determination, an appropriate state can be determined more accurately than through the motion analysis using, for example, an acceleration sensor. In addition, by performing both state determination and vital data detection from single measurement data, such as a cardiac potential signal, a measuring device can be made compact and easy to use. Furthermore, by making the device or system compact, the stress or load imposed on a measurer can be reduced, and the measurement can be performed in a more natural way.

Note that in step S110 illustrated in FIG. 14, the CPU 310 may search the power spectrum distribution obtained through the frequency analysis for the largest peak of the power spectrum in an arbitrarily determined frequency range (for example, a range between 0.05 Hz and 0.5 Hz). If the ratio of the integral of the power spectrum from the frequency of the peak to the frequency of its half-value to the whole is greater than or equal to a determined threshold value, it may be determined that the respiratory rate is in a measurable state. More specifically, it is only required to determine whether the largest peak in an arbitrarily determined frequency range (for example, a range between 0.05 Hz to 0.5 Hz) of the power spectrum distribution is more prominent than other power spectra. Accordingly, the CPU 310 may determine that the respiratory rate is in a “measurable state” by using another method.

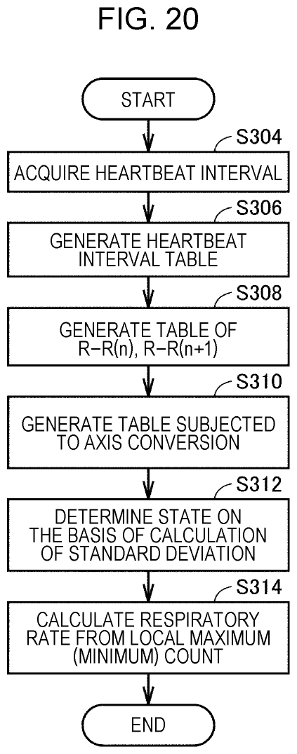

Alternatively, as illustrated in FIG. 20, the CPU 310 may determine, on the basis of a Poincare plot of the heartbeat intervals, that a target creature is in a resting state if the standard deviation or one of the product of the standard deviations and the square root of the product is greater than a predetermined value (steps S302 to 312). Thereafter, if it is determined that the state is a “measurable state”, the CPU 310 may calculate, as the respiratory rate, the number of local maximum (or minimum) points in the time-series variations of the heartbeat interval, as illustrated in FIG. 15. The CPU 310 performs the calculation illustrated in FIG. 20 at predetermined time intervals, for example, at several minute intervals, and accumulates the result of calculation in the database in the memory 320 to generate a diagnostic graph (described below).

### Technique for Outputting Diagnostic Graph

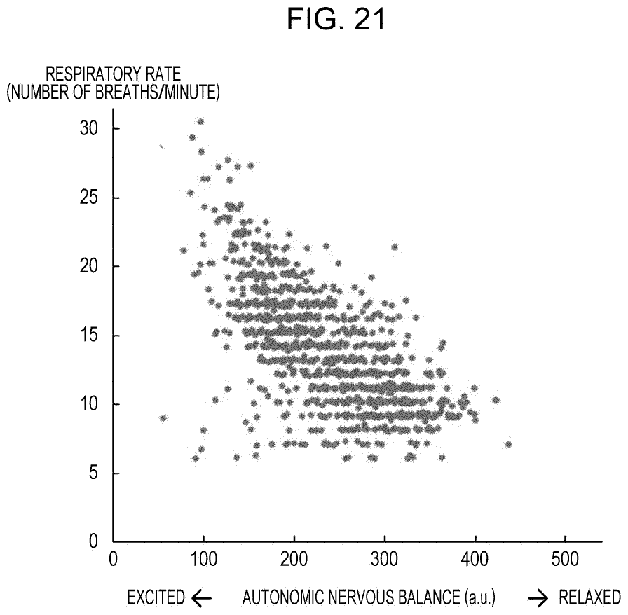

As described above, according to the present embodiment, the CPU 310 of the diagnostic terminal 300 causes the display 330 to display a variety of diagnostic graphs on the basis of the signal acquired by the signal processing device 500 and illustrated in FIG. 4. For example, as illustrated in FIG. 21, the CPU 310 displays a diagnostic graph having an abscissa axis of a numerical value representing the autonomic nervous balance and an ordinate axis of a numerical value representing the respiratory rate on the basis of a numerical value representing the autonomic nervous balance and the respiratory rate calculated at a plurality of time points, for example, at one minute intervals.

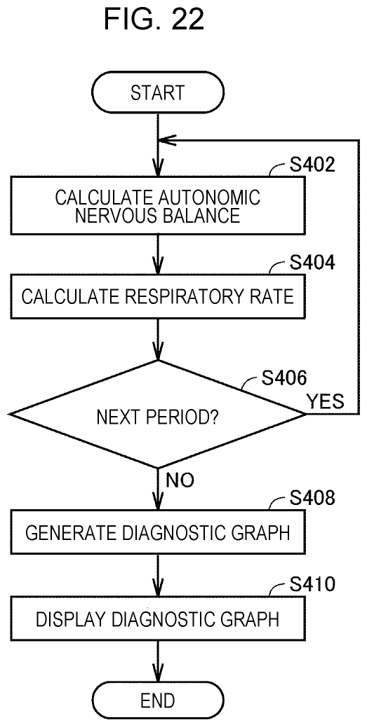

More specifically, upon receiving a specified diagnosis period, for example, several hours or several days, for a target individual creature on the basis of a program stored in the memory 320, the CPU 310 performs a process illustrated in FIG. 22. The CPU 310 calculates a numerical value representing the autonomic nervous balance calculated through the processing illustrated in FIG. 3 or 12 at predetermined time intervals, for example, at one minute intervals during the diagnosis period. Thereafter, the CPU 310 stores the numerical value in a database of the diagnostic terminal 300 or an external database (step S402). The CPU 310 calculates a numerical value representing the respiratory rate calculated through the processing illustrated in FIG. 14 or FIG. 20 at predetermined time intervals for the target individual creature. Thereafter, the CPU 310 accumulates the numerical value in the database of the diagnostic terminal 300 or the external database (step S404). If the CPU 310 completes the calculation of the numerical values indicating the autonomic nervous balance and the numerical values indicating the respiratory rate corresponding to the plurality time intervals during the diagnosis period (NO in step S406), the CPU 310 plots data of the combinations of the two numerical values on a graph having an abscissa axis of a numerical value representing the autonomic nervous balance and an ordinate axis of a numerical value representing the respiratory rate (step S408). The CPU 310 causes the display 330 to display the graph (step S410).

### Second Embodiment

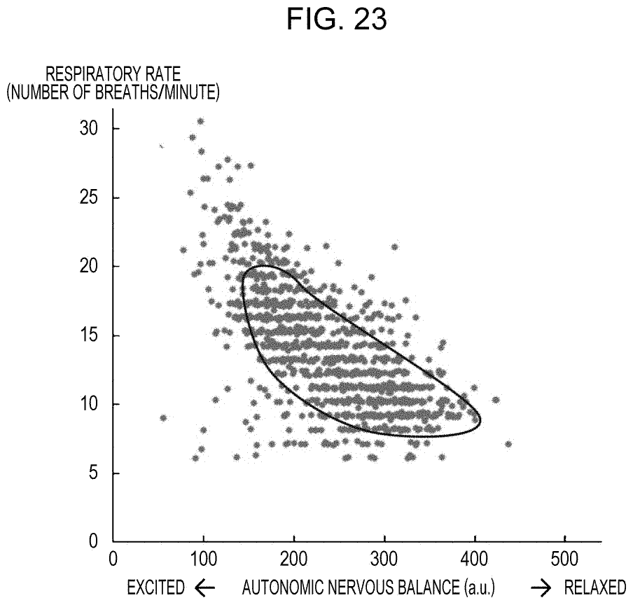

It is desirable that in addition to displaying the plots of the numerical value representing the autonomic nervous balance and the plots of the numerical value representing the respiratory rate corresponding to a plurality of time intervals, the CPU 310 display, on the diagnostic graph, an image to make the density of the plots easy to understand, as illustrated in FIG. 23. According to the present embodiment, in step S408, the CPU 310 calculates and draws contour lines relating to the density of the plots on the basis of the combinations of a numerical value representing the autonomic nervous balance and a numerical value representing the respiratory rate corresponding to the plurality of predetermined time intervals, for example, several minute intervals in accordance with the program in the memory 320. In this way, even a veterinarian who is not used to using the graph can easily understand the state of the target individual creature.

Note that it is only required to make a veterinarian to easily detect an area including a large number of the plots. A technique for drawing the contour lines may be an existing technique. That is, the technique is not limited to any particular technique.

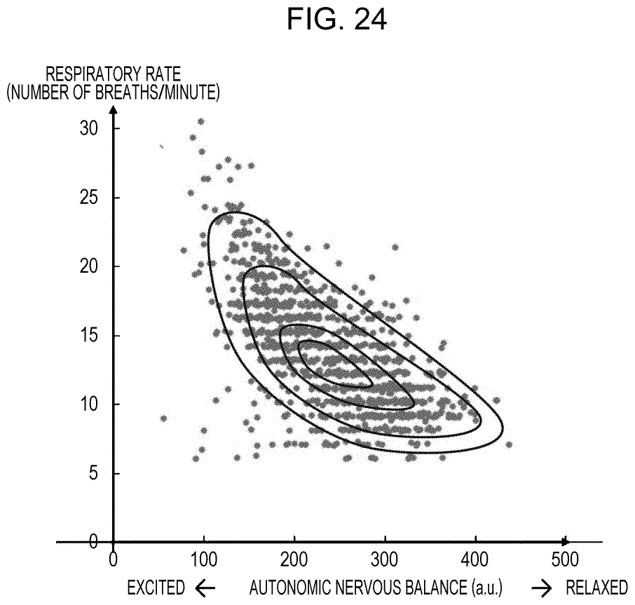

In addition, in step S408, as illustrated in FIG. 24, the CPU 310 may calculate and draw a plurality of levels of contour lines relating to the density of plots on the basis of combinations of a numerical value representing the autonomic nervous balance and a numerical value representing the respiratory rate corresponding to a plurality of predetermined time intervals, for example, several minute intervals. In this way, even a veterinarian who is not used to using the graph can more easily understand the state of the target individual creature.

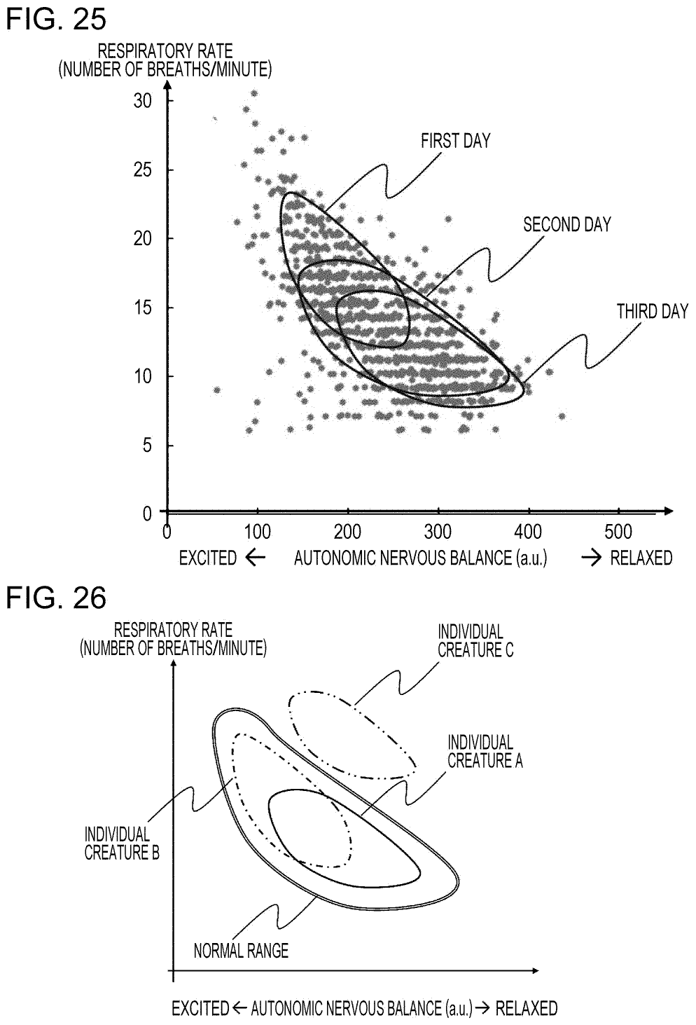

Furthermore, as illustrated in FIG. 25, in step S408, the CPU 310 may calculate and draw contour lines relating to the density of plots on the basis of the combinations of a numerical value representing the autonomic nervous balance and a numerical value representing the respiratory rate corresponding to a plurality of second predetermined time intervals, for example, several minute intervals for each of a plurality of predetermined first intervals, for example, at one day intervals during the plotted period.

For example, the CPU 310 performs the following processing in step S408. That is, the CPU 310 plots a combination of a numerical value representing the autonomic nervous balance and a numerical value representing the respiratory rate corresponding to a plurality of predetermined time intervals, for example, several minute intervals, for a measurement period extending over a plurality of days. Thereafter, the CPU 310 calculates and draws a contour line relating to the density of the lot of the first day on the basis of the combinations of a numerical value representing the autonomic nervous balance and a numerical value representing the respiratory rate corresponding to a plurality of predetermined time intervals, for example, several minute intervals, on the first day. Similarly, the CPU 310 calculates and draws a contour line relating to the density of the plot of the second day on the basis of the combinations of a numerical value representing the autonomic nervous balance and a numerical value representing the respiratory rate corresponding to a plurality of predetermined time intervals, for example, several minute intervals, on the second day. The CPU 310 calculates and draws a contour line relating to the density of the plot of the third day on the basis of the combinations of a numerical value representing the autonomic nervous balance and a numerical value representing the respiratory rate corresponding to a plurality of predetermined time intervals, for example, several minute intervals, on the third day. In this way, the veterinarian can recognize the set of plot when the target individual creature is in a stable state. As a result, the veterinarian can easily detect the state of the individual creature more accurately.

Note that the plots and the contour lines relating to different time periods may have different line types or line colors and different dot types or dot colors.

### Third Embodiment

It is desirable that in addition to displaying the plots of a numerical value representing the autonomic nervous balance and a numerical value representing the respiratory rate corresponding to a plurality of time intervals, the CPU 310 display, on the diagnostic graph, a range indicating the standard of the combination of a numerical value representing the autonomic nervous balance and a numerical value representing the respiratory rate for determining the psychological state and the physical state, as illustrated in FIG. 26. According to the present embodiment, in step S408, the CPU 310 calculates and draws, on the graph, the dot indicating the combination of a numerical value representing the autonomic nervous balance and a numerical value representing the respiratory rate and a normal range prestored in the memory 320 in a superimposed manner in accordance with a program in the memory 320. In this way, even a veterinarian who is not used to using a diagnostic graph can more easily understand the state of the target individual creature. In addition to displaying the normal range on the diagnostic graph, a range indicating that the subject is in a relaxed state and a range indicating that the subject is in an excited state may be displayed on the diagnostic graph as a criterion for determining the psychological state of the subject. Furthermore, a range indicating that the subject may have a specific disease, such as a circulatory disease, may be illustrated as a criterion for determining the physical condition of the subject. Still furthermore, the psychological state or physical state of the subject may be displayed in several stages. For example, the possibility that the subject has a specific disease may be displayed on the diagnostic graph in several stages.

### Fourth Embodiment

Furthermore, as illustrated in FIG. 26, in step S408, the CPU 310 may plot, on a diagnostic graph, combinations of a numerical value representing an autonomic nervous balance and a numerical value representing a respiratory rate corresponding to a plurality of predetermined time intervals for each of a plurality of individual creatures.

Alternatively, as illustrated in FIG. 26, in step S408, the CPU 310 may plot, for a plurality of individual creatures, the states of the individual creatures on the basis of combinations of a numerical value representing an autonomic nervous balance and a numerical value representing a respiratory rate corresponding to a plurality of predetermined time intervals and draw a contour line corresponding to each of the individual creatures.

For example, in step S408, the CPU 310 performs the following processing. That is, the CPU 310 plots, for a plurality of individual creatures, combinations of a numerical value representing the autonomic nervous balance and a numerical value representing the respiratory rate corresponding to a plurality of predetermined time intervals, for example, several minute intervals. Thereafter, the CPU 310 calculates and draws a contour line relating to the density of the plot for a first creature on the basis of the combinations of a numerical value representing the autonomic nervous balance and a numerical value representing the respiratory rate of the first creature. Similarly, the CPU 310 calculates and draws a contour line relating to the density of the plot for a second creature on the basis of the combinations of a numerical value representing the autonomic nervous balance and a numerical value representing the respiratory rate of a second creature. The CPU 310 calculates and draws a contour line relating to the density of the plot for a third creature on the basis of the combinations of a numerical value representing the autonomic nervous balance and a numerical value representing the respiratory rate of a third creature.

Even in this case, it is desirable that the CPU 310 display the normal range on the graph in a superimposed manner. For example, a veterinarian can determine that the individual creature A is healthy from the graph illustrated in FIG. 26. In addition, the veterinarian can determine that the individual creature B is healthy although the individual creature B has a high respiratory rate, because the individual creature B is frequently in an excited state for some reason. In contrast, the individual creature C may have a circulatory disease, because the individual creature C is out of the normal range and has a high respiratory rate.

Note that the plots and contour lines relating to different time periods may have different line types or line colors and different dot types or dot colors.

In addition, it is desirable that the CPU 310 switch between the display of the plot and the contour lines, the display of only the plot, and the display of only the contour lines on the basis of an instruction from a user, such as a veterinarian.

### Fifth Embodiment

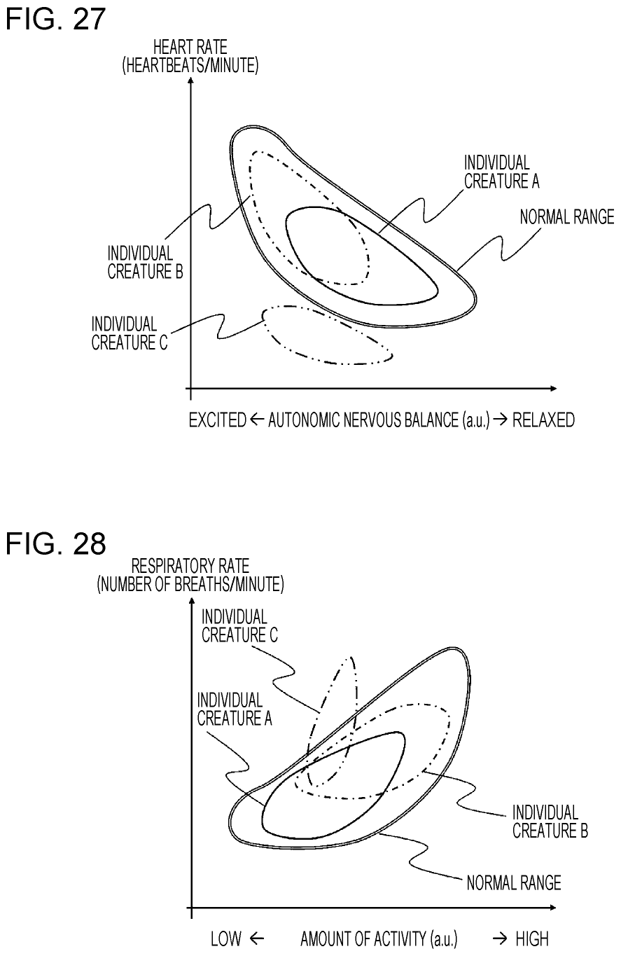

As might be expected, instead of displaying, on a graph of a numerical value representing the autonomic nervous balance and a numerical value representing the respiratory rate, an image of a plot of data obtained for each of periods, the CPU 310 may cause the display 330 to display the image on a graph having an abscissa axis of a numerical value representing the autonomic nervous balance and an ordinate axis of a numerical value representing the heart rate in accordance with a program in the memory 320, as illustrated in FIG. 27. In the case illustrated in FIG. 27, for the individual creature A and the individual creature B, the heart rate is output in accordance with the autonomic nervous balance. For this reason, the heart rates are normal. In contrast, for the individual creature C, the heart rate is low, as compared with the autonomic nervous balance. For this reason, it can be determined that the individual creature has suspected bradycardia.

Alternatively, as illustrated in FIG. 28, the CPU 310 may cause the display 330 to display, on a graph having an abscissa axis representing the amount of activity and an ordinate axis representing the respiratory rate, an image of a plot of data obtained for each of periods in accordance with a program in the memory 320. In the case illustrated in FIG. 28, for the individual creature A and the individual creature B, the respiratory rate increases with increasing amount of activity. For this reason, the respiratory rates are normal. In contrast, for the individual creature C having a respiratory rate that excessively increases with increasing amount of activity, some sort of respiratory disease is suspected. Note that the amount of activity is defined as the variances of the accelerations of parts of an individual creature acquired by the acceleration sensors attached to the parts of the individual creature. However, the amount of activity is not limited thereto.

In addition, the numerical value representing the autonomic nervous balance is not limited to the standard deviation of the Poincare plot or the product of the standard deviations. The average of the distances between two contiguous Poincare plots may be used, the numerical value representing the dispersion of the Poincare plot may be used, or another calculation method other than the Poincare plot may be used.

Furthermore, according to the above embodiment, the heartbeat interval is calculated by using the electrodes 401, 402, and 403 for acquiring the cardiac potential. However, the present invention is not limited to such an embodiment. For example, pulse wave signals may be acquired by using a photoplethysmographic pulse wave meter or a photoplethysmographic pulse oximeter, and the heartbeat interval may be calculated from the pulse wave signals. In this case, it is desirable that a part at which the pulse wave is measured be a part where the skin is exposed to outside, such as the tongue and the ear. Alternatively, heart sound signals may be acquired by using an electronic stethoscope or the like, and the heartbeat interval may be calculated from the heart sound signals. In these cases, measurement can. be performed using a method that does not use electrodes. The pulse wave signals may be acquired by using a pulse wave acquisition sensor, such as a microwave Doppler sensor, and the heartbeat interval may be calculated from the pulse wave signals. For example, a form is conceivable in which a microwave transmitting device is mounted on a ceiling or the like, and a pulse wave is acquired from a creature, such as a dog, in a noncontact manner. In this case, non-contact measurement is available, which has the effect of further reducing the load imposed on a subject.

### Sixth Embodiment

In the information processing system 1 according to the above-described embodiments, the signal processing device 500 acquires a heartbeat interval on the basis of the cardiac potential signals from the electrodes 401, 402, and 403, and the diagnostic terminal 300 calculates the information for determining the state of a creature or the information regarding the result of determination of the state of the creature from the heartbeat interval and outputs the information. However, all or some of the functions of one of the devices may be performed by another device or may be shared by a plurality of devices. Conversely, a single device may play all or some of the roles of the plurality of devices, or another device may play the roles.

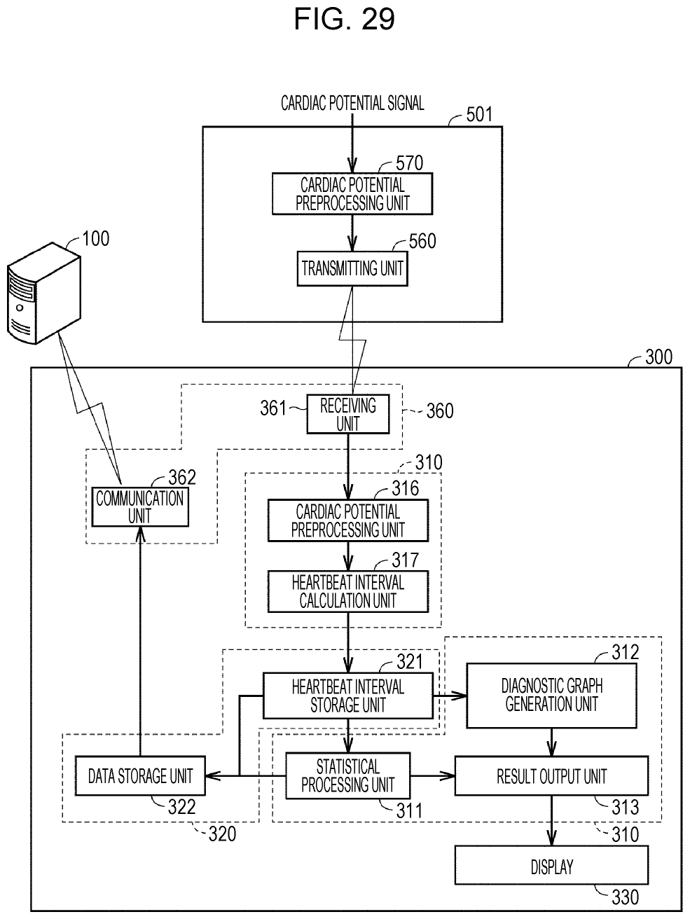

For example, as illustrated in FIG. 29, the diagnostic terminal 300 may have all or some of the functions of the signal processing device 500. In this case, the diagnostic terminal 300 acquires, from a simplified signal processing device 501, the cardiac potential signals output from the electrodes 401, 402, and 403 through wireless communication. The cardiac potential signals output from the electrodes are converted into digital signals by a simplified cardiac potential preprocessing unit 570 including only a minimum of devices (a filter device, an amplifying device, and an A/D conversion device) and are transmitted from the transmitting unit 560. The diagnostic terminal 300 calculates information for determining the heartbeat interval and the state of the creature or information indicating the result of determination of the state of the creature from the cardiac potential signals. Thereafter, the diagnostic terminal 300 outputs information regarding the final result to a display or a loudspeaker.

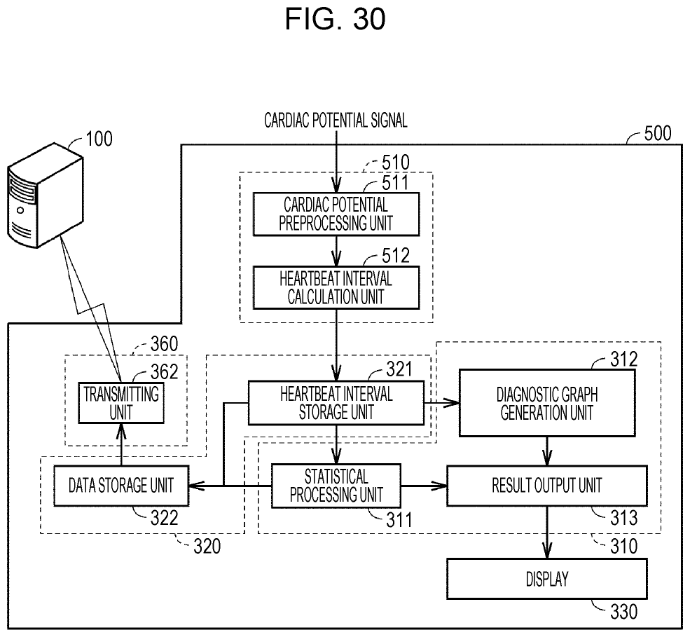

Alternatively, as illustrated in FIG. 30, the signal processing device 500 may have all or some of the functions of the diagnostic terminal 300. In this case, the signal processing device 500 calculates the information for determining the heartbeat interval and the state of the creature or the information indicating the result of determination of the state of the creature on the basis of the cardiac potential signals output from the electrodes 401, 402, and 403. Thereafter, the signal processing device 500 outputs information regarding the final result to a display or a loudspeaker.

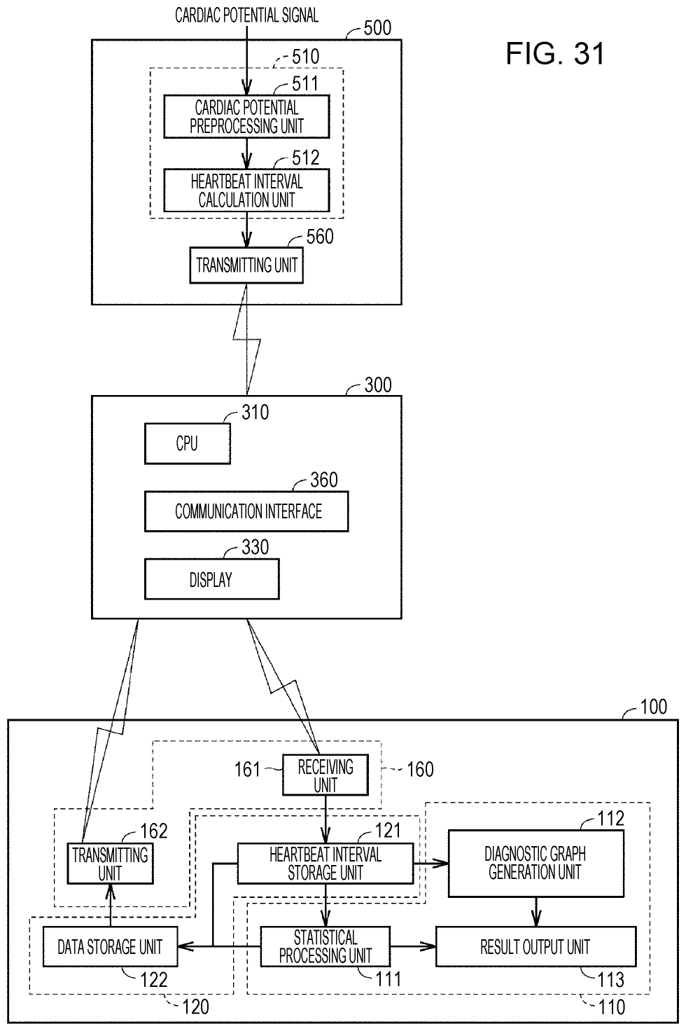

Still alternatively, as illustrated in FIG. 31, the server 100 may play the role of the diagnostic terminal 300. In this case, the server 100 has the functions of the diagnostic terminal 300 according to the above embodiments. For example, a communication terminal serving as the diagnostic terminal 300 transmits necessary information, such as the heartbeat interval received from the signal processing device 500, to the server 100 via a router, a carrier network, the Internet, or the like. The server 100 calculates the information for determining the heartbeat interval and the state of the creature or the information indicating the result of determination of the state of the creature and transmits the information to the diagnostic terminal 300. The diagnostic terminal 300 outputs information regarding the final result to a display loudspeaker.

Note that in this case, a receiving unit 161 and a transmitting unit 162 of the server 100 are naturally implemented by a communication interface 160 of the server 100. In addition, a heartbeat interval storage unit 121 and a data storage unit 122 are implemented by a memory 120 of the server 100 or another device accessible to the server 100. A statistical processing unit 111, a diagnostic graph generation unit 112, and a result output unit 113 are implemented by a CPU 110 executing a program in the memory 120.

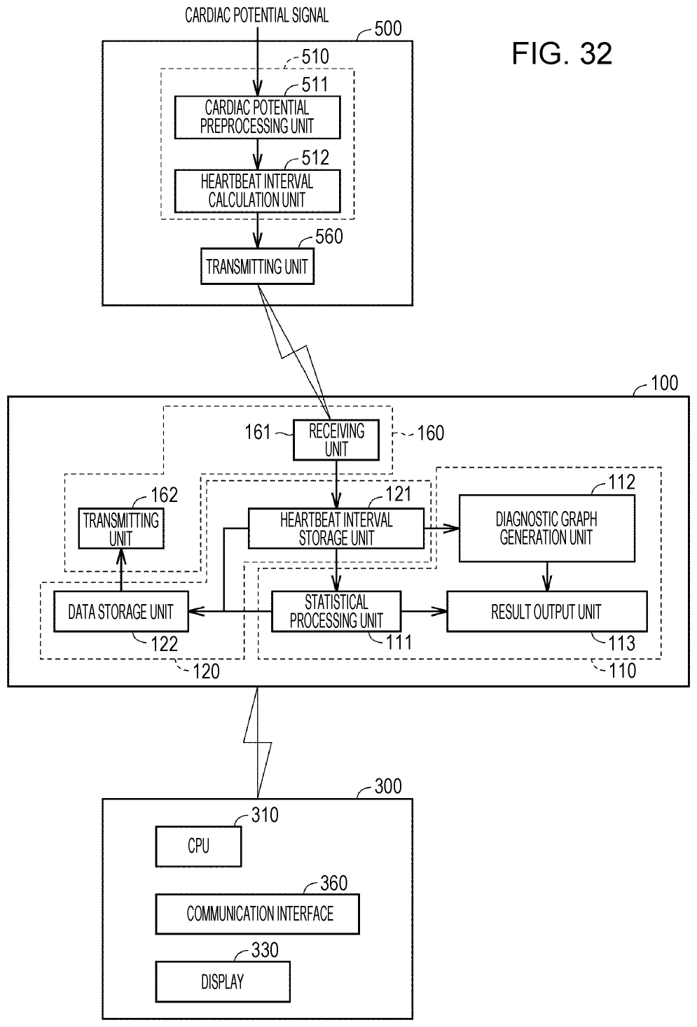

Alternatively, as illustrated in FIG. 32, the signal processing device 500 transmits necessary information, such as a heartbeat interval, to the server 100 via a router, a carrier network, the Internet, or the like. The server 100 calculates the information for determining the state of the creature or the information indicating the result of determination of the state of the creature. Thereafter, the server 100 outputs the information to a communication terminal serving as the diagnostic terminal 300 via the Internet, a carrier network, a router, or the like. The diagnostic terminal 300 outputs information regarding the final result to a display or a loudspeaker. In this case, the signal processing device 500 and the diagnostic terminal 300 need not be connected to each other via a wireless LAN or a wired LAN.

Note that even in this case, the receiving unit 161 and the transmitting unit 162 of the server 100 are naturally implemented by the communication interface 160 of the server 100. In addition, the heartbeat interval storage unit 121 and the data storage unit 122 are implemented by the memory 120 of the server 100 or another device accessible to the server 100. The statistical processing unit 111, the diagnostic graph generation unit 112, and the result output unit 113 are implemented by the CPU 110 executing a program in the memory 120.

In the description of the above embodiments, the processing for making a “Poincare plot” and the processing for performing “axis conversion after Poincare plot processing” are mentioned. However, it should be noted that the processing is not limited to printing of the image of a Poincare plot on a paper medium or displaying of the image of a Poincare plot on a display actually performed by the CPU of the diagnostic terminal 300/the server 100/the signal processing device 500. The process is the concept including, for example, a process in which the CPU stores data substantially representing a Poincare plot in a memory and loads the data into the memory.

### Other Examples of Application

It should be clearly understood that the present disclosure is also applicable to the case where the present disclosure is implemented by supplying a program to a system or an apparatus. Thus, the effect of the present disclosure can also be obtained by supplying, to a system or an apparatus, a storage medium (or a memory) that stores a program represented by software for achieving the present disclosure and causing a computer (or a CPU or an MPU) of the system or the apparatus to read the program code stored in the storage medium and execute the program code.

In this case, the program code itself read from the storage medium provides the functions of the above-described embodiments and, therefore, the storage medium that stores the program code constitutes the present disclosure.

In addition, the following case is also encompassed within. the present disclosure. That is, the functions of the above-described embodiments can be realized by not only a computer executing the readout program code but, for example, the OS (operating system), which is running on the computer, performing some or all of the actual processes on the basis of the instructions of the program code.

Furthermore, the following case is encompassed within the present disclosure. That is, after the program code read from the storage medium is written to a function expansion board inserted into a computer or another storage medium provided in a function expansion unit connected to a computer, the functions of the above-described embodiments can be realized by, for example, the function expansion unit or a CPU of the function expansion unit performing some or all of the actual processes on the basis of the instructions of the program code.

### Overview

According to the above embodiment, an information processing device is provided that includes the display 330 and a processor 310 configured to acquire vital data regarding a creature and cause the display 330 to display an image in which data regarding the creature at a plurality of time points are plotted on a graph having an abscissa axis and an ordinate axis one of which represents an autonomic nervous balance of the creature and the other represents a numerical value based on the vital data of a type that differs from the autonomic nervous balance of the creature.

Preferably, the processor 310 causes the display 330 to display a range serving as a criterion for determining one of a psychological state and a physical state of the creature together with the graph.

Preferably, the processor 310 causes the display 330 to display a normal range for the type of the creature together with the graph.

Preferably, the processor 310 causes the display 330 to display a contour line indicating the density of the plot together with the graph.

Preferably, the processor 310 causes the display 330 to display a contour line indicating the density of the plot for each of predetermined time periods together with the graph.

According to the above-described embodiments, a state acquisition program is provided that causes the processor 310 to perform a step of acquiring vital data regarding a creature, a step of calculating a numerical value representing an autonomic nervous balance of the creature at a plurality of time points, a step of calculating a numerical value based on the vital data of a type that differs from the autonomic nervous balance of the creature at the plurality of time points, and a step of causing the display 330 to display an image in which data regarding the creature at the plurality of time points are plotted on a graph having an abscissa axis and an ordinate axis one of which represents the autonomic nervous balance of the creature and the other represents a numerical value based on the vital data of the type that differs from the autonomic nervous balance of the creature.

According to the above embodiments, as illustrated in FIG. 31 and FIG. 32, in the above embodiments, the server 100 is provided that includes the communication interface 160 for communicating with the output device 300 and the processor 110 configured to acquire, via the communication interface 160, vital data regarding a creature and cause the output device 300 to output an image in which data regarding the creature at a plurality of time points are plotted on a graph having an abscissa axis and an ordinate axis one of which represents an autonomic nervous balance of the creature and the other represents a numerical value based on the vital data of a type that differs from the autonomic nervous balance of the creature.

According to the above embodiments, as illustrated in FIGS. 31 and 32, in the above embodiments, an information processing method for use of the server 100 is provided. The information processing method includes a step of receiving vital data regarding a creature, a step of calculating a numerical value representing an autonomic nervous balance of the creature at a plurality of time points, a step of calculating a numerical value based on the vital data of a type that differs from the autonomic nervous balance of the creature at the plurality of time points, and a step of causing the output device 300 to display an image in which data regarding the creature at the plurality of time points are plotted on a graph having an abscissa axis and an ordinate axis one of which represents the autonomic nervous balance of the creature and the other represents a numerical value based on the vital data of the type that differs from the autonomic nervous balance of the creature.

The embodiments disclosed herein are to be considered in all respects only as illustrative and not restrictive. The scope of the invention is, therefore, indicated by the appended claims rather than by the foregoing description. All changes which come within the meaning and range of equivalency of the claims are to be embraced within their scope.

### REFERENCE SIGNS LIST

1 information processing system
100 server
110 CPU
111 statistical processing unit
112 diagnostic graph generation unit
113 result output unit
120 memory
121 heartbeat interval storage unit
122 data storage unit
160 communication interface
161 receiving unit
162 transmitting unit
300 diagnostic terminal
310 CPU
311 statistical processing unit
312 diagnostic graph generation unit
313 result output unit
320 memory
321 heartbeat interval storage unit
321A correspondence relation table
322 data storage unit
330 display.
360 communication interface
361 receiving unit
362 transmitting unit
370 loudspeaker
401 electrode
402 electrode
403 electrode
500 signal processing device
501 simplified signal processing device
511 cardiac potential preprocessing unit
512 heartbeat interval calculation unit
560 transmitting unit
570 simplified cardiac potential preprocessing unit

1. An information processing device comprising:
a display; and
a processor configured to acquire vital data regarding a creature and cause the display to display an image in which data regarding the creature at a plurality of time points are plotted on a graph having an abscissa axis and an ordinate axis one of which represents an autonomic nervous balance of the creature and another represents a numerical value based on the vital data of a type that differs from the autonomic nervous balance of the creature.

2. The information processing device according to claim 1, wherein the processor causes the display to display a range serving as a criterion for determining one of a psychological state and a physical state of the creature together with the graph.

3. The information processing device according to claim 1, wherein the processor causes the display to display a normal range for the type of the creature together with the graph.

4. The information processing device according to claim 1, wherein the processor causes the display to display a contour line indicating a density of the plot together with the graph.

5. The information processing device according to claim 1, wherein the processor causes the display to display a contour line indicating a density of the plot for each of predetermined time periods together with the graph.

6. A state acquisition program comprising a program code for causing a processor to perform:
a step of acquiring vital data regarding a creature;
a step of calculating a numerical value representing an autonomic nervous balance of the creature at a plurality of time points;
a step of calculating a numerical value based on the vital data of a type that differs from the autonomic nervous balance of the creature at the plurality of time points; and
a step of causing a display to display an image in which data regarding the creature at the plurality of time points are plotted on a graph having an abscissa axis and an ordinate axis one of which represents the autonomic nervous balance of the creature and another represents a numerical value based on the vital data of the type that differs from the autonomic nervous balance of the creature.

7. (canceled)

8. An information processing method for use of a server, comprising:
a step of receiving vital data regarding a creature;
a step of calculating a numerical value representing an autonomic nervous balance of the creature at a plurality of time points;
a step of calculating a numerical value based on the vital data of a type that differs from the autonomic nervous balance of the creature at the plurality of time points; and
a step of causing an output device to display an image in which data regarding the creature at the plurality of time points are plotted on a graph having an abscissa axis and an ordinate axis one of which represents the autonomic nervous balance of the creature and another represents a numerical value based on the vital data of the type that differs from the autonomic nervous balance of the creature.

9. The information processing device according to claim 1, wherein
the numerical value based on the vital data of a type that differs from the autonomic nervous balance of the creature, is a respiratory rate.

10. The information processing device according to claim 1, wherein
the numerical value based on the vital data of a type that differs from the autonomic nervous balance of the creature, is a heart, rate.
